(19) United States
(12) Patent Application Publication
(10) Pub. No.: US 2017/0296608 A1
(43) Pub. Date: Oct. 19, 2017

(54) COMPOSITION OF DECREASING THE ACCUMULATION OF FAT AND USE THEREOF

(71) Applicant: Hsiu-Hsia LIN, Taipei City (TW)

(72) Inventors: Hsiu-Hsia LIN, Taipei City (TW); Chang-Yeu LIU, Taipei City (TW)

(21) Appl. No.: 15/132,595

(22) Filed: Apr. 19, 2016

(51) Int. Cl. A61K 36/53; A23K 20/10; A23K 10/16; A61K 35/742; A61K 9/00

(52) U.S. Cl. CPC A61K 36/53; A61K 35/742; A61K 9/0056; A23K 10/16; A23K 20/10; A23V 2002/00

(57) ABSTRACT

The present invention provides a natural composition of reducing weight gains and decreasing the accumulation of fat, comprising Bacillus subtilis BS139 (Storage ID: NRRL NO.B-50347) and Coleus forskohlii extract. Moreover, this composition can be used in the treatment of obesity, helping them healthy reduce fat accumulation, to achieve the effect of weight loss.

### FIELD OF THE INVENTION

The present invention provides a natural composition comprising Coleus forskohlii extract and Bacillus subtilis BS139 (the storage ID is NRRL NO. B-50347). The user could achieve the goal of weight loss healthily while taking the natural composition. Meanwhile, the user could evaluate the metabolite rate to achieve the goal of weight loss.

### BACKGROUND OF THE INVENTION

Obesity is the most important health problem in the modern society and is the most serious modern civil disease. There are lots of factors could cause obesity, such as body activities decrease, malnutrition, excessive starches and sugars intake, or excessive fat intake, etc. Wherein excessive fat intake is the most important factor. Moreover, people now changing their diet style and prefer to eat at outside, so that people often over intake unnecessary fat. Even some people select the food carefully, the invisible fats in all kinds of fresh food and artificial food is therefore unavoidable. Besides, people usually live with a busy work life, and become comfortable and relaxed state after work at home, this would cause fat accumulation in the body easily in the long run and losing weight would become more difficult to cause healthy body at risk.

Studies show that there is about 75-80% people in a civilized society population would be overweight or obese in their lifetime. Obesity would not only cause appearance defects, and also induce psychological and social obstacles and further affect the work ability; besides, it is easy to cause some physiologically symptoms, such as edema, cardiac hypertrophy, fatty liver, cholelithiasis and urolithiasis, musculoskeletal disorders, gynecologic cancers such as breast cancer or uterine cancer, gout and hyperuricemi, hyperlipidemia, angina, diabetes, hypertension, stroke or infarction and so on. According to the latest statistics from the Ministry of Health and Welfare (Taipei City, Taiwan R.O.C.), overweight or obese of adult men has reached 51% in our country, and, overweight or obese of adult women also reached 36%. In primary school, the ratio of overweight or obese in children is, 29% for boy and 21.0% for girl. Similarly, according to the latest statistics from WHO, 39% of adults aged 18 years and over were overweight and 13% were obese in worldwide. These data shows that the rate of overweight and obesity continues to rise. In addition, in the top ten causes of death, eight of the top ten causes of death are associated with obesity, such as cancer, heart disease, cerebrovascular disease, diabetes, chronic lower respiratory diseases, hypertension, chronic liver disease and cirrhosis, chronic kidney disease, etc. Therefore, it is important to maintain a healthy body weight for a personal goal to most people.

There are all kinds of dazzling, strange ways to lose weight, but some of them are not healthy ways to lose weight. The best way is to rely on regular exercises and changing diet. However, people are often lack of patience in losing weight, so that his/her biggest problem is that he/she cannot keep exercise and often cannot refuse to delicious foods. That is why the weight losing product is the best choice for the people who want to lose weight. Some common weight losing products, such as weight losing drug approved by the Ministry of Health and Welfare—phenyl propanolamine could promote the body metabolic rate, increase calorie consumption and reduce appetite, etc., and could also cause weight loss effectively in a short time. However the accompanied side effects such as palpitations, vomiting, insomnia and other side effects would happen, some people would have severe anorexia. Although the users achieve the goal of weight loss successfully, he/she would also lose his or her health.

In other words, most people would only focus on reducing the body “weight” instead of reducing the body “fat” when they carry on losing weight. That is, after losing weight, the whole body fat even the abdominal fat might not be changed. However, only the lower triglyceride level represents the effectiveness of losing weight. Therefore, providing a safe, healthy weight losing function agent without effecting the daily life, to improve existing method is an urgent mission for people who is overweight and who wants to maintain a health, low body fat percentage.

### SUMMARY OF THE INVENTION

According to above description, the present invention provides a natural composition comprising Coleus forskohlii extract and Bacillus subtilis BS139. The user could achieve the goal of losing weight healthily while taking the natural composition. Meanwhile, the user could evaluate the metabolite rate to achieve the goal of losing weight.

The present invention provides a composition for reducing fat accumulation, which comprises Coleus forskohlii extract and Bacillus subtilis BS139, wherein the Bacillus subtilis BS139 is stored in Agricultural Research Service (NRRL), the storage ID is NRRL NO. B-50347. The present invention composition for reducing fat accumulation is composed by Coleus forskohlii extract and Bacillus subtilis BS139 with a specific ratio.

The Bacillus subtilis of the present invention is stored in Agricultural Research Service (NRRL), the address is: Jamie L. Whitten Building 1400 Independence Ave., S. W. Washington D.C., 20250, the storage number is NRRL NO. B-50347, and the classification name is Bacillus subtilis and the storage time is Feb. 1, 2010.

In one example of the present invention, the content of Coleus forskohlii extract is 0.01 wt % to 0.05 wt %, and the content of Bacillus subtilis BS139 is 0.02 wt % to 0.08 wt %.

In one example of the present invention, the natural composition could reduce 20% to 40% rate of body weight gain in an animal or a human body.

In one example of the present invention, the natural composition could reduce 10% to 80% weight of abdominal fat in an animal or a human body effectively.

In one example of the present invention, the natural composition could reduce 5% to 12% Triglyceride in an animal or a human body effectively.

In one example of the present invention, the natural composition could reduce 15% to 25% caloric intake rate in an animal or a human body effectively.

In one example of the present invention, the natural composition is applied to a pharmaceutical product, health food, health-care food, food or animal feed.

The present invention also provide a preparation method of a pharmaceutical composition for treating obesity by a plant extract, wherein the plant extract comprising: effective amount of 0.01 wt % to 0.05 wt % Coleus forskohlii extract and effective amount of 0.02 wt % to 0.08 wt % Bacillus subtilis BS139, wherein the Bacillus subtilis BS139 is stored in Agricultural Research Service (NRRL), the storage ID is NRRL NO.B-50347.

### BRIEF DESCRIPTION OF THE DRAWINGS

FIG. 1 Illustration of the weight change of female rats after feeding the different feeds in different days.

FIG. 2 Illustration of the weight change of female rats after feeding the different feeds in different weeks.

FIG. 3 Illustration of the feed intake of female rats after feeding the different feeds in different weeks.

FIG. 4 Illustration of the feed efficiencies of female rats after feeding the different feeds.

### DETAILED DESCRIPTION OF THE INVENTION

Experimental Animal and Feeding Environment

Eighteen 5-weeks old Wistar female rats were used as experimental animals, each rat was 160 g and was kept in the rodent animal feeding room of Agricultural Technology Research Institute (Hsinchu City, Taiwan R.O.C). The temperature of the rodent animal feeding room was controlled in 23° C. (20-26° C.) and the light cycle was 12-hours lighted. The experimental rats were kept in cages, which were sterilized by high temperature and high pressure; besides, the feeding water was provided with water bottles, which were also sterilized by high pressure.

The Construction of Different Groups of Wistar Female Rats Feeding with Different Feeds

Two rats were kept in one cage, and the animal numbers, cage numbers were labeled. The six-week experiment was followed by 7-days habitual period. The rats were randomly into 3 groups: control group, oil-added feed group, oil-added and drug treated feed group. There were 3 cages in total, and there were 6 rats in each group. The rats in the control group were feed with imported granular rodent feed (Lab Diet® 5001 Rodent Diet). The rats in oil-added feed group was feed with 10% soybean oil containing high-fat feed, wherein the high-fat feed was prepared by adding 10% soybean oil after smashing the imported granular rodent feed and shaping into a lump. The high-fat feed was prepared two days before feeding, so as to avoid the problem of oil oxidation. The rats in the oil-added and drug treated feed group were feed with high-fat feed, and the Coleus forskohlii extract and Bacillus subtilis BS139 were then added. The main nutritious ingredients in the feeds were listed as Table 1, wherein all the data in the table were average values.

TABLE 1
Main nutritious ingredient in the feeds

| | Granular rodent | |
|---|---|---|
| | feed group | |
| | (Lab Diet ® 5001 | Oil-added |
| Group | Rodent Diet) | feed group |
| Total Calories (kcal/kg) | 4,070 | 4,560 |
| Total Proteins (%) | 23.7 | 21.8 |
| Total Fat (%) | 5.2 | 14.4 |
| Total Calcium (%) | 0.94 | 0.86 |
| Total Phosphorous (%) | 0.68 | 0.60 |

Experiment Analysis

The rats were weighted in the beginning of the experiment (Day 1) and every week respectively, and the weekly feed consumption of each rats were recorded. The daily clinical symptoms were observed during experiment, and whether the clinical symptoms or death were existed in each animal was recorded. The tail vein blood was collected in the end of experiment (Day 42), and the glucose level, triglyceride level, aspartate aminotransferase (AST) level, and alanine aminotransferase (ALT) level of each rats were tested by Automated Clinical Chemistry Analyzer (Kodak Ektachem DT-II System, Rochester, N.Y., USA). Then the rats were sacrificed for pathological observation, and the tissue weight was recorded.

### Example 1. The Intake Amounts of Feeds and Calories

Please refer to the Table 2, the feed intake amounts were slightly different between different groups during experiment period, but there was no statistical difference (P>0.05), wherein the values in Table 2 were average values.

TABLE 2
The intake amounts of feeds (g/rat/week) and calories (kcal/rat)

| | | | | | | | Total | Total calories |
|---|---|---|---|---|---|---|---|---|
| | | | | | | | feed | intake amount |
| | Week | Week | Week | Week | Week | Week | intake | (calculated |
| groups | 1 | 2 | 3 | 4 | 5 | 6 | amount | value) |
| Control group | 151 | 185 | 185 | 209 | 204 | 198 | 1,132 | 4,607 |
| Oil-added feed | 138 | 134 | 210 | 224 | 244 | 218 | 1,168 | 5,326 |
| group | | | | | | | | |
| oil-added and drug | 177 | 134 | 192 | 194 | 262 | 183 | 1,142 | 5,207 |
| treated feed group | | | | | | | | |

To compare the rodent feed group with control group, the oil-added feed group was feed with 10% soybean oil containing high-fat feed. In the oil-added feed group, the feed intake amount was 8.6% lower than control group during week 1, the amount was even 27.6% lower than control group during week 2; however the feed intake amount was higher than control group afterward, the amount was increased 7-19% during week 3 and week 6. The feed intake amount of the oil-added feed group was decreased during week 1 and week 2, which seemed to be affected by the added oil; however, after two weeks habituation, the feed intake amounts during following 4 weeks were higher than control group. During experiment period, the total feed intake amount of oil-added group was similar to the total feed intake amount of control group; however, the total calories intake amount was 15.6% higher than control group, which was matched with the purpose of high-fat feed to increase the rat's calories intake amount.

The rats in the oil-added and drug treated feed group were then feed with Coleus forskohlii extract and Bacillus subtilis BS139, and the intake amount was measured, the test result of intake amount change in this group is not consistent with the test result of oil-added group. During week 1, the rat's feed intake amount of oil-added and drug treated feed group was 17.2% higher than the control group, and was 28.3% higher than the oil-added feed group. During week 2, the rat's feed intake amount of oil-added and drug treated feed group was similar to the rats in oil-added feed group, but 27.6% lower than the rats in control group. Then, during the week 5, the rat's feed intake amount of oil-added and drug treated feed group was 28.4 higher than the rats in control group. During week 4 and week 6, the rat's feed intake amount of oil-added and drug treated feed group was both 7% lower than the rats in control group. The added Coleus forskohlii extract and Bacillus subtilis BS139 seem to inhibit the rat's feed intake amount after one week (that is, week 2, 4, 6). During the whole experiment period, the rat feed intake amount of oil-added and drug treated feed group was similar to rats in control group, however, the total calories intake amount was 13% higher than the rats in control group but 2.6% lower than the rats in the oil-added feed group.

### Example 2: Growth Curve and Weight Changes

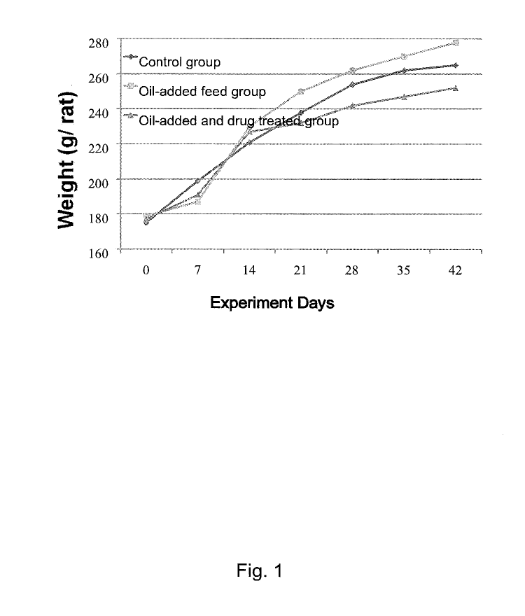

Please refer to the FIG. 1, all the rats in different groups were similar to each other in weight, which is about 117 g in the beginning; however, all the rats in different groups gain weight as more feed days. The rats in the control gain weight gradually slow down as more feed days, and the growth curve of the oil-added feed group was different with the control group. It's noteworthy that the rat's weight of the oil-added and drug treated feed group was lower than control group and oil-added feed group from day 21, the difference between the oil-added and drug treated feed group and control group, oil-added feed group was increase as more feed days but the increase of the difference was not equidistant.

Please refer to the Table 3, all the data in the Table 3 were average values, the superscripts showed the significant differences in the same row. Wherein the P value of a and b was smaller than 0.05, the P value of a and c was smaller than 0.01, and the P value of a and d was smaller than 0.001.

TABLE 3
Weekly Weight gain (g/rat)

| | | | | | | | Total |
|---|---|---|---|---|---|---|---|
| | Week | Week | Week | Week | Week | Week | weight |
| Group | 1 | 2 | 3 | 4 | 5 | 6 | gain |
| Control | 24.2^{a} | 22.2^{a} | 16.5^{a} | 16.5 | 7.7 | 3.2 | 90.2^{a} |
| group | | | | | | | |
| Oil-added | 8.0^{b} | 42.3^{d} | 20.2^{a} | 12.5 | 7.8 | 7.7 | 98.5^{a} |
| feed group | | | | | | | |
| oil-added | 14.3^{ab} | 35.2^{cd} | 5.2^{c} | 10.3 | 4.7 | 5.2 | 74.8^{b} |
| and drug | | | | | | | |
| treated feed | | | | | | | |
| group | | | | | | | |

The rats in the control group gained weight over 22 g during week 1 and 2, gained weight about 16.5 g during week 3 and 4, gained 7.7 g during week 5, and gained only 3.2 g during week 6. The rats in oil-added feed group gained only 8 g during week 1, which is significantly lower than control group (P<0.05). On the other hand, the gained weight in oil-added feed group was significantly higher than control group during week 2, which is 42.3 g (P<0.001). Then, the weekly weight gain in control group and oil-added feed group was no significant difference between two groups during week 3-6. The weight gain in oil-added and drug treated feed group was no difference to the other two groups during week 1, but the weight gain in oil-added and drug treated feed group was significantly higher than control group during week 2, which is 35.2 g (P<0.01). During week 3, the rats in oil-added and drug treated feed group only gain 5.2 g, which was significantly lower than control group and oil-added feed group (P<0.01). Then, the weekly rate of weight gain in oil-added and drug treated feed group was no difference to the other groups in statics, but the values of weight gain in oil-added and drug treated feed group was lower than in control group and oil-added feed group during week 4-6. During the experiment period, the total weight gain in control group and oil-added feed group was 90.2 g and 98.5 g respectively. In contrast, the oil-added and drug treated feed group only gain 74.8 g, which was significantly lower than the other groups (P<0.05). The result showed that the weight curve of oil-added and drug treated feed group was lower than the control group and oil-added feed group since week 3, which prove that the feed of the composition of 0.01 wt % to 0.05 wt % Coleus forskohlii extract and 0.02 wt % to 0.08 wt % Bacillus subtilis BS139 could help reduce weight gain.

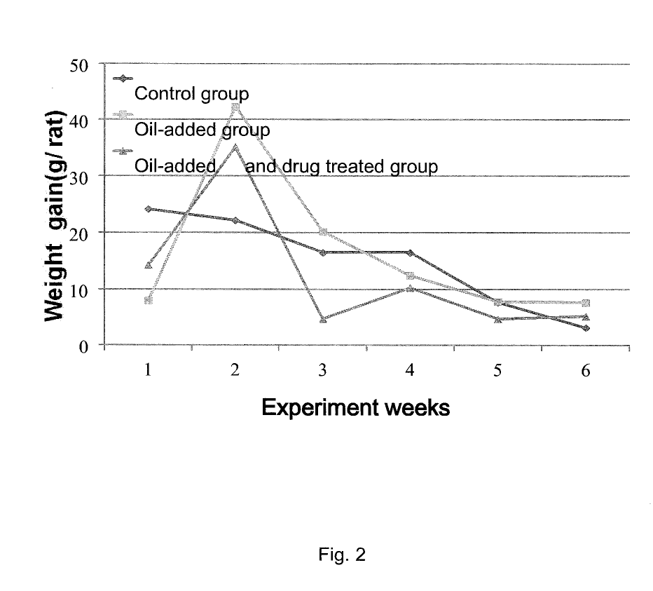

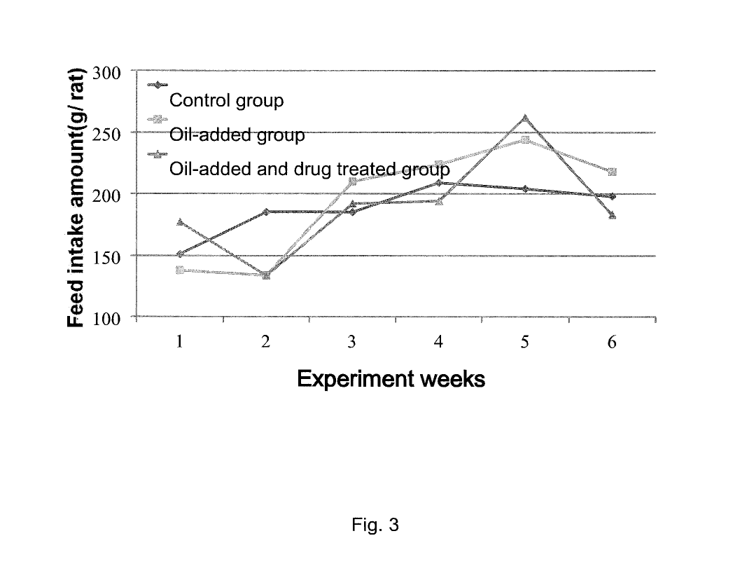

Please refer to FIG. 2 and FIG. 3, the rat in the group of high-fat feed, the amount of feed intake could be reduced due to accommodation in early stage, so as to reduce the weight gain of the rat (week 1). However, the amount of feed intake in oil-added and drug treated feed group was 17% higher than the control group, but the weight gain was 40% lower than the control group, which represented that the added Coleus forskohlii extract and Bacillus subtilis BS139 composition seemed to have the function of inhibit the weight gain. Except for week 2, similar result was observed during week 3, 4, 5 and 6. In comparison with control group, the amount of feed intake in oil-added and drug treated feed group was 3.8% higher, but the weight gain was 68.5% lower during week 3; the amount of feed intake in oil-added and drug treated feed group was 28.4% higher, but the weight gain was 38.7% lower during week 5. In comparison with oil-added group, the amount of feed intake in oil-added and drug treated feed group was 8.6% higher, but the weight gain was 74.3% lower during week 3. Furtherly, the amount of feed intake in oil-added and drug treated feed group was 7.4% higher, but the weight gain was 39.7% lower during week 5. The result showed that the amount of feed intake was increased in the oil-added and drug treated feed group, but the increase rate of weight gain was lower than control group and oil-added feed group. That is, the composition of 0.01 wt % to 0.05 wt % Coleus forskohlii extract and 0.02 wt % to 0.08 wt % Bacillus subtilis BS139 in the present invention could reduce 20% to 40% rate of body weight gain in an animal or a human body.

### Example 3. Tissue Weight

All the animals were healthy and not dead, nor observed any clinical symptoms during experiment period. Please refer to Table 4, all the data in the Table 4 were average values, the superscripts showed the significant differences of statics in the same row. Wherein the P value of a and b was smaller than 0.05, the P value of a and c was smaller than 0.01. The heart and kidney weight was no difference between all the groups after animal sacrificed. However, the abdominal fat weight of oil-added feed group was 58.5% higher (P<0.05) than control group; but the abdominal fat weight of oil-added and drug treated feed group was 44.5% lower (P<0.01) than oil-added feed group, and was 12.1% lower (P>0.05) than control group, while the rats in oil-added and drug treated feed group were also feed with high-fat feed. The Coleus forskohlii extract and Bacillus subtilis BS139 composition was added in the oil-added and drug treated feed group, which inhibit the abdominal fat accumulation under similar calories intake condition. The abdominal fat accumulation in the oil-added and drug treated feed group was significantly lower than control group and oil-added feed group, which proved that the Coleus forskohlii extract and Bacillus subtilis BS139 composition of the present invention could reduce the fat intake and reduce the fat accumulation effectively. That is, the composition of 0.01 wt % to 0.05 wt % Coleus forskohlii extract and 0.02 wt % to 0.08 wt % Bacillus subtilis BS139 in the present invention could reduce 10% to 80% weight of abdominal fat accumulation in an animal or a human body.

TABLE 4
Tissue weight (g)

| | | | Oil-added |
|---|---|---|---|
| | Control | Oil-added | and drug treated |
| Tissue | group | feed group | feed group |
| Heart | 0.86 | 0.88 | 0.90 |
| Kidney | 2.42 | 2.44 | 2.26 |
| Abdominal fat | 3.23^{b} | 5.12^{a} | 2.84^{bc} |

### Example 4. Biochemical Profile of Blood

Please refer to Table 5, all the data in the Table 5 were average values. The superscripts showed the significant differences of statics in the same row, for example, the P value of a and c was smaller than 0.01. In comparison with control group, the blood glucose level was lower (P<0.01) in the oil-add feed group, which means that the high-fat feed could reduce the blood glucose level, but the triglycerate level was slightly higher (16.5%). The blood glucose level in oil-added and drug treated feed group was lower (P<0.01) than control group; but the increase level of triglyceride (5.5%) in oil-added and drug treated feed group was 11% lower than oil-added feed group. Therefore, the added Coleus forskohlii extract and Bacillus subtilis BS139 composition could reduce the triglycerate when the rats eat more high-fat food, wherein the mechanism might not be related to glucose generation, but related to lipid metabolism and generation. That is, the composition of 0.01 wt % to 0.05 wt % Coleus forskohlii extract and 0.02 wt % to 0.08 wt % Bacillus subtilis BS139 in the present invention could reduce 5% to 12% triglyceride in blood either an animal or a human body.

TABLE 5
Biochemical Profile of Blood

| | | | Oil-added |
|---|---|---|---|
| | | | and drug |
| | Control | Oil-added | treated |
| Item | group | feed group | feed group |
| Glucose (mg/dL) | 232^{a} | 146^{c} | 151^{c} |
| Triglycerate (mg./dL) | 69.7 | 81.2 | 73.5 |
| aspartate aminotransferase | 160 | 135 | 93.2 |
| (AST) | | | |
| alanine | 41.3 | 49.5 | 40.3 |
| aminotransferase(ALT) | | | |

Besides, the aspartate aminotransferase (AST) level and alanine aminotransferase (ALT) level were important markers to evaluate the effectiveness of liver disease treatment, which were often used to evaluate the liver cell damage level and classify the liver disease as acute or chronic. Aspartate aminotransferase (AST) abundantly existed in liver, and alanine aminotransferase (ALT) exists in heart, liver and muscle cell. When these cells were damaged, especially when hepatic cells were damaged, the activity of aspartate aminotransferase (AST) and alanine aminotransferase (ALT) of the serum would increase significantly. In this example, although the aspartate aminotransferase (AST) level in oil-added feed group was lower than control group, alanine aminotransferase (ALT) level remained high. Moreover, the alanine aminotransferase (ALT) level in oil-added feed group was slightly increased, on the other side, the alanine aminotransferase (ALT) level in oil-added and drug treated feed group showed slightly decreased. However, the differences were not much between different groups.

### Example 5. Feed Efficiency

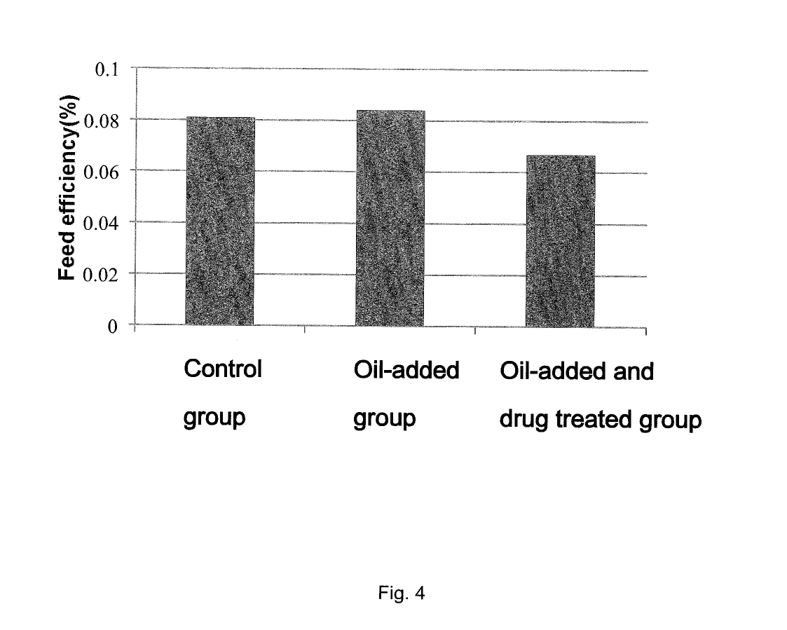

Please refer to FIG. 4, the total weight gain was divided by total intake amount, so as to calculate the efficiency of feed converted into weight. The calculated result of control group was similar to oil-added feed group. The feed efficiency of oil-added and drug treated feed group was 19% lower than the other groups, which means the rats feed with the feed containing Coleus forskohlii extract and Bacillus subtilis BS139 needed to eat 19% more feed to achieve similar weight gain. Therefore, the Coleus forskohlii extract and Bacillus subtilis BS139 composition of the present invention could reduce the feed nutrition intake and reduce the reaction of feed convert into weight effectively. That is, the composition of the present invention could reduce 15% to 25% caloric intake rate in an animal or a human body effectively.

According to the experiment of the present invention, the Wistar female rats feed with high-fat feed would increase weight and abdominal fat accumulation; meanwhile, the Coleus forskohlii extract and Bacillus Subtilis BS139 composition of the present invention could control the high-fat feed induced rate of weight gain (24.1%), and inhibit the lipid metabolism (triglyceride) and abdominal fat accumulation (44.5%), the inhibition level is greater than the increase level of weight gain and abdominal fat accumulation in normal feed group. Besides, after 6 weeks, there is no any clinical symptoms (severe side effects) in the rats feed with the Coleus forskohlii extract and Bacillus subtilis BS139 composition and high-fat feed. The composition of the present invention could inhibit user's weight gain, reduce the fat intake, reduce the fat accumulation, without reducing the appetite and without having any side effect, so that the user could control his/her body weight in a healthy style. As mention above, the present invention could reduce the weight gain in animal or human body, reduce triglyceride in animal or human body, reduce the food intake rate in animal or human body through specific percentage range of the composition of 0.01 wt % to 0.05 wt % Coleus forskohlii extract and 0.02 wt % to 0.08 wt % Bacillus subtilis BS139 (Storage ID: NRRL NO. B-50347).

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

What is claimed is:

1. A natural composition of reducing weight gains and decreasing the accumulation of fat, comprising 0.01 wt % to 0.05 wt % Coleus Forskohlii extract and 0.02 wt % to 0.08 wt % Bacillus subtilis BS139 (Storage ID: NRRL NO.B-50347), wherein the natural composition could reduce 20% to 40% rate of body weight gain in an animal or a human body.

2. The natural composition of claim 1, the natural composition could reduce 10% to 80% weight of abdominal fat in an animal or a human body effectively.

3. The natural composition of claim 1, the natural composition could reduce 5% to 12% Triglyceride in an animal or a human body effectively.

4. The natural composition of claim 1, the natural composition could reduce 15% to 25% caloric intake rate in an animal or a human body effectively.

5. The natural composition of claim 1, the natural composition is applied to a pharmaceutical product, health food, health-care food, food or animal feed.

6. A preparation method of a pharmaceutical composition for treating obesity by a plant extract composition of, wherein the plant extract comprising: 0.01 wt % to 0.05 wt % Coleus forskohlii extract and 0.02 wt % to 0.08 wt % Bacillus subtilis BS139 (Storage ID: NRRL NO.B-50347), wherein the natural composition could reduce 20% to 40% rate of body weight gain in an animal or a human body.

7. The preparation method of claim 6, the natural composition could reduce 10% to 80% weight of abdominal fat in an animal or a human body effectively.

8. The preparation method of claim 6, the natural composition could reduce 5% to 12% Triglyceride in an animal or a human body effectively.

9. The preparation method of claim 6, the natural composition could reduce 15% to 25% caloric intake rate in an animal or a human body effectively.

10. The preparation method of claim 6, the natural composition is applied to a pharmaceutical product, health food, health-care food, food or animal feed.
